(19) United States
(12) Patent Application Publication
(10) Pub. No.: US 2023/0347155 A1
(43) Pub. Date: Nov. 2, 2023

(54) Implantable medical device operative in the presence of an MRI device

(71) Applicant: BIOTRONIK SE & Co. KG, Berlin (DE)

(72) Inventors: Thomas DOERR, Berlin (DE); Sergey ERSHOV, Berlin (DE); Torsten RADTKE, Berlin (DE); Uwe KAMENZ, Berlin (DE)

(73) Assignee: BIOTRONIK SE & Co. KG, Berlin (DE)

(21) Appl. No.: 18/014,816

(22) PCT Filed: Jun. 21, 2021
(86) PCT No.: PCT/EP2021/066797

§ 371 Date: Jan. 6, 2023

(30) Foreign Application Priority Data
Jul. 8, 2020 (EP) 20184645.8

(51) Int. Cl. A61N 1/37

(52) U.S. Cl. CPC A61N 1/3718

(57) ABSTRACT

An implantable medical device comprises a sensing device for sensing a measurement quantity indicative of a presence of an MRI device, a processing device for controlling operation of the implantable medical device and for identifying a presence of an MRI device based on measurement values obtained from the sensing device; and a program memory. An analysis module is configured to store information concerning a multiplicity of events relating to operation of the implantable medical device. The program memory is configured to store at least two program routines for operating the implantable medical device in case of a presence of an MRI device. The processing device is configured, for controlling operation of the implantable medical device in the presence of an MRI device, to select one of said at least two program routines based on a statistical analysis of information concerning a predefined number of events of said multiplicity of events.

### CROSS-REFERENCE TO RELATED APPLICATIONS

This application is the United States National Phase under 35 U.S.C. § 371 of PCT International Patent Application No. PCT/EP2021/066797, filed on Jun. 21, 2021, which claims the benefit of European Patent Application No. 20184645.8, filed on Jul. 8, 2020, the disclosures of which are hereby incorporated by reference herein in their entireties.

### TECHNICAL FIELD

The present invention relates to an implantable medical device according to the preamble of claim 1 and to a method for operating an implantable medical device.

### BACKGROUND

An implantable medical device shall be configured for implantation into a patient. An implantable medical device in this context may, for example, be a pacemaker device for providing for a pacing action in a patient's heart, e.g., a CRT device, or a defibrillator device, such as an implantable cardioverter-defibrillator (ICD), for providing for a defibrillation, a neuro-stimulation device or generally an implantable pulse generator for generating stimulation pulses.

An implantable medical device of this kind comprises a sensing device for sensing a measurement quantity indicative of a presence of an MRI device, a processing device for controlling operation of the implantable medical device and for identifying a presence of an MRI device based on measurement values obtained from the sensing device, and a program memory.

An implantable medical device, for example, a stimulation device such as a pacemaker device or a defibrillator device, generally is configured to output electrical stimulation energy for performing a therapeutic stimulation action. An implantable medical device in addition may be configured to sense electrical signals relating, e.g., to cardiac activity, in particular to trigger and clock a stimulation action. If an implantable medical device is introduced into an electromagnetic field of an MRI device, it can be assumed that electrical signals are induced on leads and other conducting parts of the implantable medical device, such electrical signals causing a disturbance of the operation of the medical device. For example, for an implantable medical device in the shape of a pacemaker device or a defibrillator device, a disturbance of a stimulation action may cause an erroneous stimulation and hence may have a significant impact on a therapy of a patient, which needs to be avoided.

There hence is a need to detect whether a patient carrying an implantable medical device is in the vicinity of an MRI device, such that, if this is the case, operation of the medical device may be modified in order to reduce a risk for a malfunction of the implantable medical device.

U.S. Pat. No. 9,364,663 B2 discloses an implantable medical device including a power supply, a sensing device and/or a stimulation device. An MR detection unit may identify an MR-typical journey of an implantable medical device on a patient bed of an MRI device, based on a change over time of measurement values of at least two magnetic field sensors and a difference between the measured values of the at least two magnetic field sensors. Herein, if measurement values of the at least two magnetic field sensors exceed a threshold, a presence of an MRI device is assumed.

U.S. Pat. No. 9,981,124 B2 discloses an implantable medical device having a first magnetic field direction sensor located at a first location within a housing and configured to generate a signal representative of a first direction of a magnetic field at the first location, and a second magnetic field direction sensor located at a second location within the housing and configured to generate a signal representative of a second direction of the magnetic field at the second location. Based on signals obtained via the first magnetic field direction sensor and the second magnetic field direction sensor it is concluded for a presence of an MRI device.

U.S. Publication No. 2011/0077706 A1 discloses an implantable medical device which automatically determines at least a portion of the parameters of an exposure operating mode based on stored information regarding sensed physiological events or therapy provided over a predetermined period of time. The implantable medical device may configure itself to operate in accordance with the automatically determined parameters of the exposure operating mode in response to detecting a destructive energy field. Alternatively, the implantable medical device may provide the automatically determined parameters of the exposure operating mode to a physician as suggested or recommended parameters for the exposure operating mode.

In case a patient carrying an implantable medical device such as a pacemaker device or a defibrillator device shall undergo an MRI examination, nowadays it is conventional practice that a patient prior to undergoing the MRI examination has to visit a physician such that the physician may adapt operation of the implantable medical device using an external programming device. For example, the physician may enable, using the external programming device, an MRI compatible operating mode in which a stimulation function of the implantable medical device is switched off or is modified in order to reduce a risk of malfunction in the presence of an electromagnetic field caused by an MRI device, for example, the MRI device's constant magnetic field, the device's gradient field or the device's RF field. The physician herein may enable the respective MRI compatible mode, such that the MRI compatible mode may be switched on once the implantable medical device is brought into proximity with an MRI device, wherein the actual activation of the MRI compatible mode may take place automatically by a suitable detection within the implantable medical device.

As the mode which is enabled by the physician may depend on an actual condition of the patient, the treatment by the physician should take place within a close time range prior to the MRI examination. Overall, a burden for the patient carrying an implantable medical device may be substantial when having to undergo an MRI examination, and additional costs may arise in the context of an MRI examination.

The present disclosure is directed toward overcoming one or more of the above-mentioned problems, though not necessarily limited to embodiments that do.

### SUMMARY

It is an object of the instant invention to provide an implantable medical device and a method for operating an implantable medical device which in an easy-to-implement way allow for a reliable operation of the implantable medical device in the presence of an MRI device, while reducing a burden for a patient in the context of an MRI examination.

At least this object is achieved by means of an implantable medical device comprising the features of claim 1.

Accordingly, the implantable medical device comprises an analysis module configured to store information concerning a multiplicity of events relating to operation of the implantable medical device, wherein the program memory is configured to store at least two program routines for operating the implantable medical device in case of a presence of an MRI device, wherein the processing device is configured, for controlling operation of the implantable medical device in the presence of an MRI device, to select one of said at least two program routines based on a statistical analysis of information concerning a predefined number of events of said multiplicity of events.

Operation of the implantable medical device is controlled by the processing device, which, for controlling the operation, uses a software program defining the mode of operation of the implantable medical device. Herein, in general operation of the implantable medical device a default operating routine may be employed for providing, e.g., a stimulation function, such as a pacemaker function of a pacemaker device, a defibrillator function of a defibrillator device or a neuro-stimulation function of a neuro-stimulation device.

In general, the default operating routine may be based, e.g., on a triggered, synchronous stimulation, involving a detection of activity, e.g., cardiac activity, such that a pacemaker function, for example, is triggered in a synchronous manner with cardiac activity. The default operating mode may, for example, be a so-called VVI mode, relating, for example, to a ventricular anti-bradycardia pacing, or a DDD mode, relating, for example, to a dual-chamber anti-bradycardia pacing.

If, by means of measurements obtained from the sensing device, the processing device identifies the presence of an MRI device, for example, due to a rise of the magnetic fields as measured by the sensing device, the processing device is configured to switch to a program routine which is specifically adapted for operation of the implantable medical device in the presence of an MRI device. Herein, in a program memory of the implantable medical device multiple, different program routines are stored, each program routine to defining a specific operation of the implantable medical device suitable for operating the implantable medical device in the presence of an MRI device.

Generally, within the presence of an MRI device, a stimulation function should be adapted such that the electromagnetic fields of the MRI device do not give rise to a malfunction of the implantable medical device. Hence, the program routines are adopted such that a stimulation function is modified and no erroneous stimulation is triggered by a potential signal induction caused by an electromagnetic field of the MRI device.

For operating the implantable medical device in the presence of an MRI device, the processing device is configured to select one of the multiple program routines stored in the program memory for operating the implantable medical device in the presence of an MRI device. The selection herein takes place based on a statistical analysis of data which is stored in the analysis module, the data representing information concerning a multiplicity of events relating to the operation of the implantable medical device.

It is to be noted that the analysis module may be implemented as software on the processing device, the analysis module providing for a data structure for storing information relating to events relating to the operation of the implantable medical device. It however is also conceivable that the analysis module is implemented by hardware, for example in the shape of a separate processor.

The analysis module, in addition to storing a data structure containing the information concerning the multiplicity of events may provide for a statistical analysis, such that the analysis module also provides for computations for outputting statistical characteristics relating to the events.

It further is to be noted that the program routines may be implemented by software and may, for example, be part of the general operating software of the processing device for controlling operation of the implantable medical device. The program routines hence may be implemented as different, e.g., modular software functions which are part of the software of the processing device.

The processing device bases its selection on a statistical analysis of a predefined number of events. The number of events which is analyzed for selection of the program routine hence is fixed, making the selection specifically reliable in that it is ensured that the selection is based on a sufficient basis of available data.

The selection of the program routine takes place once the processing device identifies the presence of an MRI device, based on measurements obtained from the sensing device, for example, based on measurements of a magnetic field strength. Hence, as soon as an MRI device is present, the processing device switches to a program routine which specifically is adapted for operation in the presence of an MRI device, such that a malfunction of the implantable medical device during an MRI examination is avoided. As soon as it is identified, again based on measurements from the sensing device, that the MRI device no longer is present, the processing device may switch back to the default operating mode, hence resuming the normal operation of the implantable medical device.

In one embodiment, the analysis module comprises a circular buffer for storing information concerning the predefined number of events. The circular buffer may, for example, have a fixed size, for example, having a fixed number of storage locations for storing information pieces, such as a number of storage locations in between 100 and 5,000, for example between 500 and 2,000, for example 1000. The circular buffer hence may store a fixed number of information pieces, wherein old information pieces are overwritten by new information pieces once the new information pieces are obtained. Hence, old information is cyclically overwritten by new information, such that a fixed number of information pieces is held within the circular buffer.

If in the beginning of operating the implantable medical device the circular buffer is not yet fully filled, in the beginning of operating the implantable medical device the analysis may be based on the data available, wherein in any case information up to the maximum size of the circular buffer is considered for the analysis, the maximum size of the circular buffer hence defining the predefined number of events which are considered for the selection of the program.

Because a predefined number of events are considered for the statistical analysis on which the selection is based, it is made sure that a sufficient basis of data for the selection is used, which may not be the case if a fixed time range is used, in particular in a situation in which in a fixed time range only a limited amount of data, for example measurement data, is available.

In one embodiment, at least some of the events of the multiplicity of events may relate to events of cardiac activity, for example, to stimulated contractions or intrinsic contractions, in the right ventricle, in the left ventricle, or in both the right ventricle and the left ventricle. The information stored in the analysis module hence relates to measurement data indicative of cardiac activity.

In one embodiment, at least some events of the multiplicity of events may relate to events of stimulation. Such stimulation may relate to a cardiac stimulation or to a neuro-stimulation. The events may in particular relate to any event in which a stimulation pulse is injected for providing for a cardiac stimulation or for a neuro-stimulation.

In one embodiment, at least some of the events relate to a triggering of a therapeutic function. If the implantable medical device is, for example, a defibrillator device, such as an ICD device, the triggering of a therapeutic function may, for example, be the triggering of a defibrillation shock. If the implantable medical device is, for example, a pacemaker device, a therapeutic function may, for example, be an anti-bradycardia pacing function, or a bi-ventricular stimulation (BiV) function for resynchronization. If the implantable medical device is a neuro-stimulation device, the therapeutic function may, for example, be a spinal cord stimulation (SCS) function or another neuro-stimulation function.

The information relating to the predefined number of events, as stored, for example, in a circular buffer, may relate to events of the same type, for example, to measurement data indicative of cardiac activity. In this case multiple circular buffers may be present to store information relating to different types of events.

Alternatively, the information relating to the predefined number of events, as stored, for example, in a circular buffer, may relate to events of different types, such that a predefined number of events of different types are considered for the statistical analysis. In this case the overall number of events which are considered is fixed for the statistical analysis, wherein the number of information pieces relating to events of a particular type, for example cardiac activity, is smaller than the overall predefined number of events. For example, if the circular buffer has a size of 1000 storage locations for storing information relating to different events, information pieces relating to, e.g., events of cardiac activity may be stored only in a fraction of the overall storage locations, other storage locations being filled by information pieces relating to other events.

In one embodiment, one of the multiple program routines represents an OFF mode in which a stimulation function is switched off. Hence, in the presence of an MRI device the processing device may select a program routine in which a stimulation function is switched off, such that no stimulation takes place in case a patient undergoes an MRI examination.

In one embodiment, another of the program routines may represent an asynchronous mode employing an asynchronous stimulation. Within such asynchronous stimulation a stimulation without a triggering based on a sensing of, e.g., cardiac activity takes place, such that an erroneous triggering is avoided due to interaction with the MRI device's electromagnetic fields.

The program routine representing the asynchronous mode may, for example, employ a so-called DOO or VOO mode, wherein also different program routines for carrying out a DOO mode and a VOO mode may be present. In a DOO mode an asynchronous atrial and ventricular pacing may take place. In a VOO mode an asynchronous ventricular pacing only takes place.

To obtain information relating to events, the processing device may be configured to initiate measurements of, for example, cardiac activity. Such test operations may be conducted cyclically, for example once a day, or for a predefined number of times a day, to for example 2 times, 3 times, 4 times or more a day, for example each hour or each half hour. Within such cyclical test operations cardiac activity, for example intrinsic conductivity, may be measured, wherein measurements for each test operation may, for example, be averaged and may yield a measurement result for one event, for example, an average heart rate at a particular time.

In one embodiment, the processing device is configured to adapt at least one parameter of any one of said at least two program routines based on a statistical analysis of information concerning the multiplicity of events. Hence, in addition to selecting a particular program routine, a particular parameter of the program routine may be adapted, such that the program routine is tailored to the actual condition of a patient. For example, based on an analysis of the heart rate as derived from the stored information relating to the predefined number of events, a stimulation frequency for an asynchronous pacing mode may be adapted and set, a stimulation amplitude may be adapted and set, a stimulation pulse width may be adapted and set, and a stimulation location, for example, for a stimulation in the right ventricle, in the left ventricle or both in the right ventricle and the left ventricle, may be determined and set.

For example, the stimulation location may be determined based on an analysis of an intrinsic RV-LV delay, and/or based on atrial-ventricular conduction times. Alternatively or in addition, the stimulation energy may be adapted based on excitation measurements. Alternatively or in addition, a DOO or VOO mode may be set in dependence of a statistical analysis of an atrial fibrillation. Yet alternatively, a maximum stimulation energy for a neuro-stimulation may be set in dependence on a documented threshold of the Phrenic nerve stimulation.

In one embodiment, the predefined number of events which is considered for the statistical analysis is programmable by a user. A user may hence set the number which is to be used and considered for selection of the program routine in the presence of an MRI device. An MRI device uses a strong, constant magnet field having, for example, a nominal magnetic field strength of above 1 T, for example 1.5 T, 3 T or 7 T. By superimposing the constant magnetic field with time-varying magnetic gradient fields a magnetic resonant effect is induced, which may be detected using RF detection coils for picking up signals within a patient's body to conduct an imaging of the patient. Herein, when a patient shall undergo an MRI examination, the patient typically is placed on a patient bed of the MRI device and, by moving the patient bed into a bore of the MRI device, is placed with a body part to be examined inside of the bore of the MRI device.

If a patient carrying an implantable medical device, such as a cardiac stimulation device, shall undergo an MRI examination, the implantable medical device shall be enabled to detect that the patient is approaching an MRI device such that operation of the implantable medical device may suitably be modified in order to reduce a risk of an impact of the MRI device, in particular the strong magnetic field of the MRI device, on the operation of the implantable medical device. The sensing device may, for example, be configured to measure a magnetic field strength at the location of the implantable medical device. In this case, for detecting whether a patient carrying an implantable medical device is in the vicinity of an MRI device, measurement values of the sensing device for detecting a magnetic field are examined. If it, for example, is found that a multiplicity of measurement values obtained from the sensing device of the implantable medical device indicate an increase of the strength of the magnetic field, it is assumed that a patient is placed on a patient bed of an MRI device and is moved, by moving the patient's bed with a substantially constant velocity, into the bore of the MRI device.

Alternatively or in addition, the sensing device may be configured to measure a gradient magnetic field sensor to enable the processing device to identify the presence of an MRI device.

Alternatively or in addition, the sensing device may be an RF field sensor for measuring an RF electromagnetic field of an MRI device to enable the processing device to identify the presence of an MRI device.

Alternatively or in addition, the sensing device may be a position sensor, a vibration sensor, or a sensor for detecting characteristic electrical signal patterns, as induced, for example, on the leads of the implantable medical device.

An object is also achieved by a method for operating an implantable medical device, the method comprising: sensing a measurement quantity indicative of a presence of an MRI device using a sensing device; identifying a presence of an MRI device based on measurement values obtained from the sensing device using a processing device for controlling operation of the implantable medical device; storing, using an analysis module, information concerning a multiplicity of events relating to operation of the implantable medical device; and selecting, based on a statistical analysis of information concerning a predefined number of events of said multiplicity of events, by the processing device in case of a presence of an MRI device one of at least two program routines for operating the implantable medical device in the presence of an MRI device, the at least two program routines being stored in a program memory.

The advantages and advantageous embodiments of the implantable medical device as described above equally apply also to the method, such that it shall be referred to the above.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Various features and advantages of the present invention may be more readily understood with reference to the following detailed description and the embodiments shown in the drawings. Herein,

FIG. 1 shows a schematic illustration of an implantable medical device in a patient;

FIG. 2 shows a schematic drawing of an MRI device;

FIG. 3 shows a schematic drawing of an implantable medical device;

FIG. 4 shows a schematic drawing of preparatory steps for conducting an MRI examination on a patient carrying an implantable medical device such as a pacemaker device; and

FIG. 5 shows a schematic drawing of an implantable medical device being configured for adapting operation in case of a presence of an MRI device.

### DETAILED DESCRIPTION

Subsequently, embodiments of the present invention shall be described in detail with reference to the drawings. In the drawings, like reference numerals shall designate functionally similar structural elements, if appropriate.

It is to be noted that the embodiments are not limiting for the present invention, but merely represent illustrative examples.

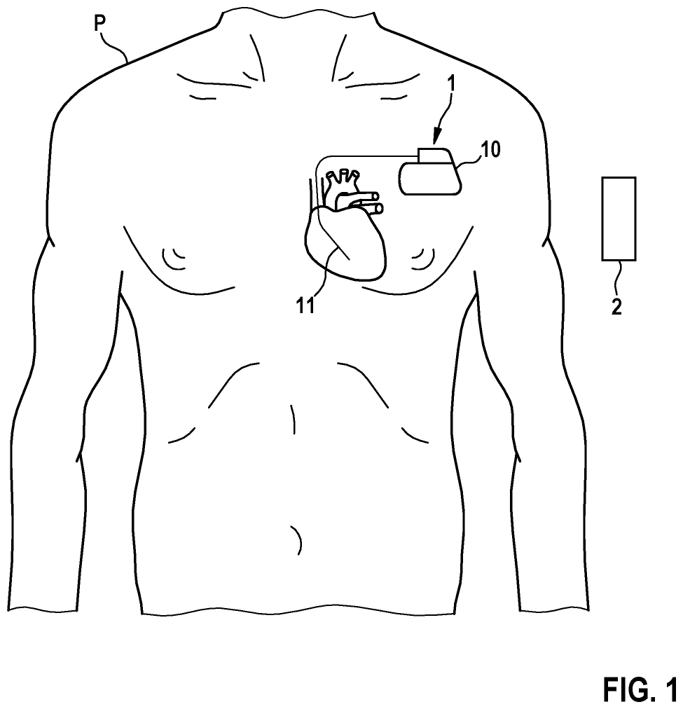

FIG. 1 shows a schematic illustration of an implantable medical device 1, for example, in the shape of a stimulation device, such as a pacing device or a defibrillation device. The implantable medical device 1 may, for example, comprise a generator 10 which, for example, as illustrated in FIG. 1, may be subcutaneously implanted into a patient, wherein an electrode 11 is connected to the generator 10 and extends from the generator 10 towards a region of interest, for example, the patient's heart, such that a therapy function may be provided at the region of interest, for example, in the patient's heart.

An implantable medical device 1 of this kind may, for example, be configured to provide a therapy function over a prolonged period of time, for example, a pacing function or a defibrillator function. The implantable medical device 1, for this, may be permanently implanted into a patient P and may function in a substantially autarkic manner, wherein a communication connection may be established with the implantable medical device 1 using an external device 2, for example, to program the implantable medical device 1 or to transfer, using, for example, telemetry, data from the implantable medical device 1 to the external device 2.

An external device 2 may communicate with the implantable medical device 1 using electromagnetic means, for example, by establishing an inductive coupling in between the implantable medical device 1 and the external device 2.

The external device 2 may alternatively be a permanent magnet which may be brought into the vicinity of the implantable medical device 1 in order to trigger an action of the implantable medical device 1, for example, for waking up the medical device 1.

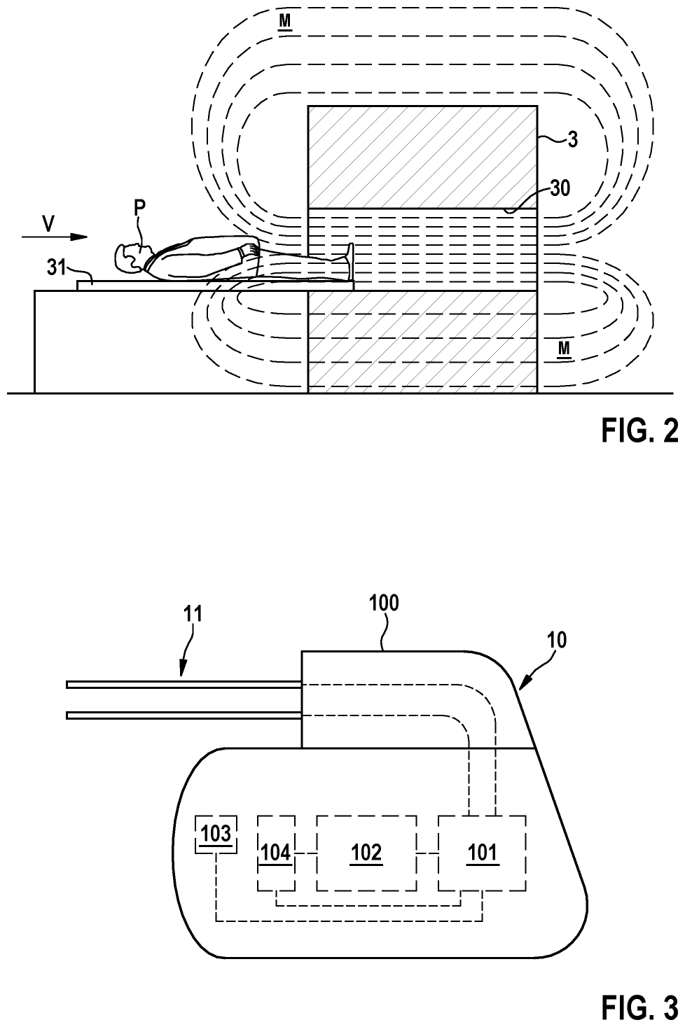

If, as schematically illustrated in FIG. 2, a patient P shall undergo an MRI examination using an MRI device 3, the patient P, carrying an implantable medical device 1, is introduced into a bore 30 of the MRI device 3 by placing the patient P on a patient bed 31 and by continuously moving, using an electro-motoric drive of the patient bed 31, the patient P into the bore 30 in a movement direction V. When moving the patient P into the bore 30 of the MRI device 3, the patient P herein is brought into the range of a constant magnetic field M of the MRI device 3, the magnetic field M generally having a maximum magnetic field strength (corresponding to the magnetic flux density B and indicated usually in Tesla [T]) within the bore 30. Hence, when the patient P is moved into the bore 30 of the MRI device 3, the magnetic field strength of the magnetic field M at the location of the implantable medical device 1 will steadily increase.

Caused by a magnetic field M of an MRI device 3 electrical signals may be induced within an implantable medical device 1. Hence, it shall be detected if an implantable medical device 1 comes into the range of an MRI device 3, such that operation of the implantable medical device 1 can suitably be modified in order to avoid a disturbance of operation by the MRI device 3.

FIG. 3 illustrates an embodiment of a generator 10 of an implantable medical device 1, for example, in the shape of a stimulation device such as a pacemaker device or a defibrillator device. Included in a housing of the generator device 10 is a processing device 101, implemented, e.g., by electronic circuitry on a circuit board, which serves to control operation of the generator 10 for transmission of electrical stimulation energy via the electrodes 11 connected to a connector block 100 of the generator 10, and for analyzing sense signals received, e.g., via the electrodes 11 to provide for a therapy aligned to an activity of, for example, the patient's heart.

The generator 10 further comprises an energy storage 102 in the shape of a battery, a sensing device 103, for example, in the shape of a GMR sensor for sensing magnetic fields, and a communication device 104 for establishing a communication connection, for example, to an external device 2, as illustrated in FIG. 1.

The sensing device 103 is connected to the processing device 101 and is configured to conduct measurements yielding measurement values indicative of a magnetic field strength at the location of the sensing device 103. The sensing device 103 may, for example, be configured to conduct measurements at a specified sampling rate, for example, at a rate in between 1 Hz and 50 Hz, for example 4 Hz. The sensing device 103 provides (discrete) measurement values to the processing device 101, which are analyzed by the processing device 101 and are used to identify the presence of an MRI device 3.

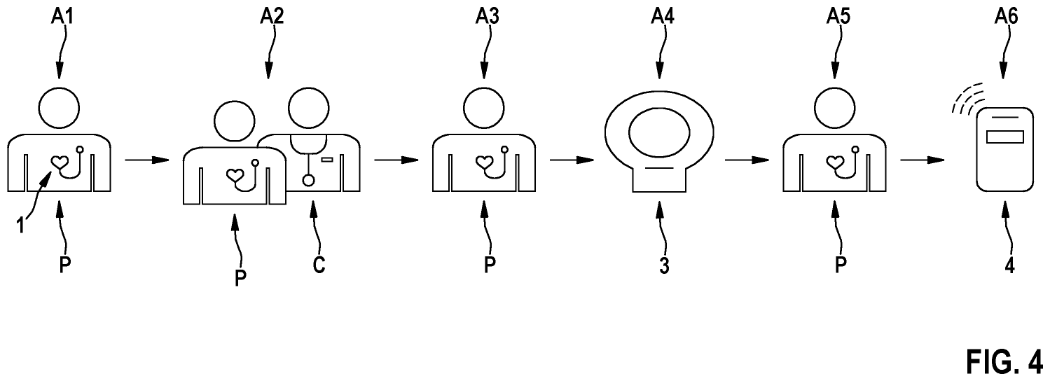

Referring now to FIG. 4, if a patient P carrying an implantable medical device 1 (for example, in the shape of a pacemaker device, such as a CRT device) has to undergo an MRI examination, the patient P typically has to visit a physician C such that the physician C may adapt operation of the implantable medical device 1 in order to avoid a malfunction of the implantable medical device 1 during MRI examination (steps A1, A2 in FIG. 4). In the course of adaption, for example, an MRI compatible operating mode is enabled in which a stimulation function of the implantable medical device 1 is switched off, or in which the stimulation function is adapted such that the implantable medical device 1 may safely operate in the presence of electromagnetic fields as caused by an MRI device 3.

Such adaption of the configuration of the implantable medical device 1 should take place within a rather short time range prior to the MRI examination, for example, within 14 days prior to the MRI examination. The patient P may then undergo the MRI examination (steps A3, A4 in FIG. 4), wherein the MRI compatible operating mode is activated once the presence of the MRI device 3 is detected by the implantable medical device 1 such that during the MRI examination the implantable medical device operates according to its modified, MRI compatible operating mode. Subsequent to the MRI examination data may be reported to a home monitoring system 4 (steps A5, A6 in FIG. 4), and the implantable medical device 1 may switch back to resume normal operation.

In a conventional scenario, a patient P hence must visit a physician C prior to an MRI examination, which must suitably configure the implantable medical device 1 in order to enable an operation adapted for a potential interaction with electromagnetic fields of an MRI device 3. This increases the burden for the patient P, and also causes additional costs.

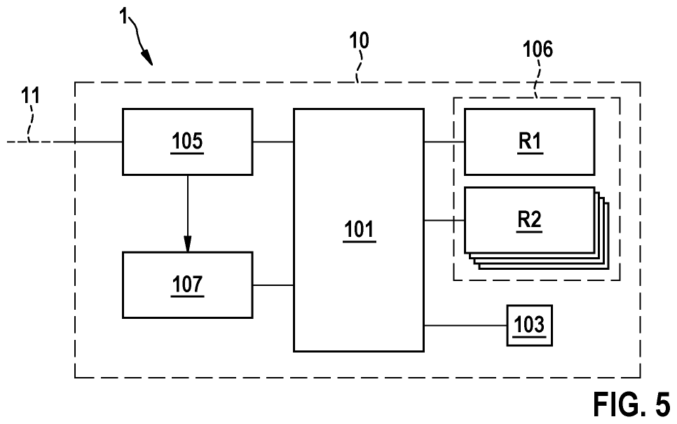

Referring now to FIG. 5, an implantable medical device 1 is adapted for an automatic change in configuration if a presence of an MRI device 3 is detected based on measurement data obtained from a sensing device 103.

The implantable medical device as shown in FIG. 5 comprises a processing device 101 which controls operation of the implantable medical device 1, in particular to control a stimulation module 105 configured to transmit stimulation energy towards electrodes 11 attached to a generator portion 10 of the implantable medical device 1 and to receive sensing data from the electrodes 11. For example, in one embodiment the implantable medical device 1 may be configured to provide for an anti-bradycardia stimulation, for which a therapeutic function is triggered once a bradycardia is detected in order to provide for a pacing counteracting the bradycardia.

Generally, in a default operating mode the processing device 101 may, for example, provide for a stimulation based on sensed measurement data, in order to, for example, provide a stimulation in a synchronous fashion in synchronicity and triggered by sensed activity. For example, in a default operating mode the processing device may be configured to operate in a so-called VVI mode, relating, for example, to a ventricular anti-bradycardia pacing, or a DDD mode, relating, for example, to a dual-chamber anti-bradycardia pacing.

In case the processing device 101 identifies, based on measurement data obtained from a sensing device 103 (such as a GMR sensor for measuring a magnetic field strength at the location of the implantable medical device 1), the presence of an MRI device 3, the processing device 101 is configured to automatically adapt operation of the implantable medical device 1. For this, the processing device 101 switches to one of a multiplicity of program routines R1, R2, which are stored in a program memory 106 and provide for modular functions which provide for a suitable operation of the implantable medical device 1 in the presence of electromagnetic fields of an MRI device 3. In particular, by means of the program routines R1, R2 it is made sure that malfunctions due to interaction with electromagnetic fields of the MRI device 3 are avoided.

In the program memory 106, different program routines R1, R2 are stored, which provide for different modes of operations. Herein, the processing device 101 is configured to select one of the program routines R1, R2 based on a statistical analysis of information relating to a predefined number of events as stored in an analysis module 107.

The analysis module 101 may, for example, comprise a circular buffer having a fixed size, such that in the analysis module 17 information relating to a predefined number of events may be stored.

The events may, for example, relate to events of cardiac activity, events of stimulation, events of a triggering of a therapeutic function, or other events, such as programming events.

Information relating to events of cardiac activity may, for example, be measurement data indicating an intrinsic or stimulated cardiac activity, such as an atrial or ventricular activity. Such measurement data may, for example, be obtained in test operations, which are performed cyclically, for example, in regular intervals once a day, twice a day, 3 times a day, 4 times a day, each hour, or each half hour. During a measurement period herein, for example, the heart rate and/or other characteristics of cardiac activity may be obtained, such as an amplitude, a pulse width, a pulse energy or the like, wherein the respective characteristic value may be averaged over the measurement period, such that, for example, an averaged heart rate at different time instances per day is obtained.

Information relating to events of stimulation may, for example, indicate a triggering of a specific stimulation, such as a defibrillation shock, or a neuro-stimulation.

Information relating to events of a triggering of a therapeutic function may, for example, indicate whether a specific function, such as an anti-bradycardia stimulation, a biventricular stimulation for resynchronization, an SCS stimulation or another neuro-stimulation has been triggered.

Based on the statistical analysis it in particular may be determined whether and what type of simulation may be required for a particular patient P during an MRI examination.

One of the program routines R1, R2 may, for example, represent an OFF mode in which a stimulation function is switched off, such that, if the implantable medical device 1 is operated according to the particular program routines R1, R2, no stimulation takes place once the patient P is in the vicinity of an MRI device 3.

Another of the program routines R1, R2 in turn may represent an asynchronous mode in which a stimulation takes place, but in an asynchronous fashion, i.e., without sensing data and without basing a stimulation, such as a pacing action, on sensed data. The asynchronous mode may, for example, be a VOO or a DOO mode.

The processing device 101 may, for example, be configured to choose between the to program routines R1, R2 based on an analysis whether a particular patient P requires a stimulation or not during the time period of an MRI examination. If it, for example, is found based on the statistical analysis that a patient P requires a pacing action even during the MRI examination, the processing device 101 may switch to a program routine R1, R2 which represents an asynchronous mode.

If in contrast it is found based on the statistical analysis that no stimulation is necessarily required during the time of the MRI examination, the processing device 101 may switch to a program routine R1, R2 which represents the OFF mode, such that stimulation is switched off during the MRI examination. If it is, for example, found, based on the statistical analysis, that the heart rate has been stable for the patient over recent measurements, requiring, e.g., no anti-bradycardia stimulation, it may be switched to the OFF mode.

In addition to selecting a particular program routine R1, R2, the processing device 101 may be configured to adapt and program a particular parameter of a particular program routine R1, R2, such as a stimulation energy/amplitude, a stimulation frequency, a stimulation location (for example, in the right ventricle, in the left ventricle, or in both the right ventricle and the left ventricle), a particular mode of operation (DOO vs. VOO mode) or the like.

The selection of the program routine R1, R2 takes place once the processing device 101, based on readings of the sensing device 103, identifies the presence of an MRI device 3.

The sensing device 103 herein may, for example, be configured to measure a magnetic field strength for identifying, for example, a rise of the magnetic field strength. Alternatively, the sensing device 103 may be adapted to measure a gradient field vector, an RF field, a position, a vibration, or a characteristic electrical signal pattern as, for example, induced on the electrodes 11. Multiple sensors measuring different measurement quantities may be used in combination.

Once the processing device 101 identifies that the patient P no longer is in the presence of an MRI device 3, the processing device 101 switches back to the default operating mode, by, for example, switching on a stimulation function or switching to a synchronous mode.

By means of the processing device 101, hence, an automatic switching of operation to a program routine R1, R2 which is suitable for use during an MRI examination is provided. This makes it possible for a patient P to undergo an MRI examination without specific preparation, in particular without having to visit a physician C (as shown in FIG. 4) for the purpose of configuring the implantable medical device 1 prior to undergoing the MRI examination. The burden for the patient P hence is reduced, while at the same time saving time and costs.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device (pacemaker device)
10 Generator
100 Connector block
101 Processing device
102 Energy storage
103 Sensing device
104 Communication module
105 Stimulation module
106 Program memory
107 Analysis module
11 Electrode
2 External device
3 MRI device
30 Bore
31 Patient bed
4 Home monitoring system
A1-A6 Steps
C Clinician
M Magnetic field (magnetic flux density)
P Patient
R1, R2 Program routine
V Moving direction

1. An implantable medical device, comprising: a sensing device for sensing a measurement quantity indicative of a presence of an MRI device a processing device for controlling operation of the implantable medical device and for identifying a presence of an MRI device based on measurement values obtained from the sensing device; program memory; and an analysis module configured to store information concerning a multiplicity of events relating to operation of the implantable medical device, wherein the program memory is configured to store at least two program routines for operating the implantable medical device case of a presence of an MRI device, wherein the processing device is configured, for controlling operation of the implantable medical device in the presence of an MRI device, to select one of said at least two program routines based on a statistical analysis of information concerning a predefined number of events of said multiplicity of events.

2. The implantable medical device of claim 1, wherein the analysis module comprises a circular buffer for storing information concerning said predefined number of events.

3. The implantable medical device of claim 1, wherein at least some events of said multiplicity of events relate to events of cardiac activity.

4. The implantable medical device of claim 3, wherein events of cardiac activity are events of stimulated contractions or intrinsic contractions.

5. The implantable medical device of claim 1, wherein at least some events of said multiplicity of events relate to stimulation events.

6. The implantable medical device of claim 5, wherein stimulation events are events of a cardiac stimulation or a neuro-stimulation.

7. The implantable medical device of claim 1, wherein at least some events of said multiplicity of events relate to a triggering of a therapeutic function.

8. The implantable medical device of claim 1, wherein one of said at least two program routines represents an OFF mode in which a stimulation function is switched off.

9. The implantable medical device of claim 1, wherein one of said at least two program routines represents an asynchronous mode employing an asynchronous stimulation.

10. The implantable medical device of claim 1, wherein the processing device is configured to control the implantable medical device for cyclically conducting test operations for obtaining information concerning events relating to the operation of the implantable medical device.

11. The implantable medical device of claim 1, wherein the processing device is configured to adapt at least one parameter of any one of said at least two program routines based on a statistical analysis of information concerning said multiplicity of events.

12. The implantable medical device of claim 1, wherein said a predefined number of events is programmable by a user.

13. A method for operating an implantable medical device comprising: sensing a measurement quantity indicative of a presence of an MRI device using a sensing device; identifying a presence of an MRI device based on measurement values obtained from the sensing device using a processing device for controlling operation of the implantable medical device; storing, using an analysis module, information concerning a multiplicity of events relating to operation of the implantable medical device; and selecting, based on a statistical analysis of information concerning a predefined number of events of said multiplicity of events, by the processing device in case of a presence of an MRI device one of at least two program routines for operating the implantable medical device in the presence of an MRI device, the at least two program routines being stored in a program memory.
